(19) United States
(12) Patent Application Publication
(10) Pub. No.: US 2019/0224171 A1
(43) Pub. Date: Jul. 25, 2019

(54) LIQUID RIZATRIPTAN COMPOSITIONS

(71) Applicant: Insys Development Company, Inc., Chandler, AZ (US)

(72) Inventors: Ravi Vaishya, Chandler, AZ (US); Huaguang Li, Chandler, AZ (US); Oscar Liu, Short Hills, NJ (US); Venkat R. Goskonda, Phoenix, AZ (US)

(21) Appl. No.: 16/251,545

(22) Filed: Jan. 18, 2019

Related U.S. Application Data
(60) Provisional application No. 62/620,055, filed on Jan. 22, 2018.

(51) Int. Cl. A61K 31/4196; A61K 9/08; A61K 47/10; A61K 47/40; A61K 47/26; A61P 25/06

(52) U.S. Cl. CPC A61K 31/4196; A61K 9/08; A61K 47/10; A61K 9/0043; A61K 47/26; A61P 25/06; A61K 47/40

(57) ABSTRACT

The present invention is directed to liquid rizatriptan compositions. The present invention is further directed to methods of treating migraines comprising administering a liquid rizatriptan composition to a subject in need thereof.

### FIELD OF THE INVENTION

The present invention is related to liquid rizatriptan compositions. The present invention is further related to methods of treating migraines comprising administering a liquid rizatriptan composition to a subject in need thereof.

### BACKGROUND OF THE INVENTION

There are more than 3 million cases of migraines diagnosed in the United States each year. Migraines can be a serious condition causing pain for several hours to several days. This pain may be so severe that the person suffering the migraine is unable to perform daily tasks. In some migraine sufferers, a phenomenon known as an aura may occur prior to the onset of the migraine. An aura may consist of flashes of light, blind spots, tingling on one side of the face or one extremity, an unpleasant smell or confusing thoughts or experiences. Even with the commonness and severity of migraines, very little is known about their cause or pathology.

Current treatment focuses on either pain relief or prevention. Preventative medications include antidepressants, anti-seizure drugs, beta-blockers and botulinum toxin. However, not all migraines are able to be prevented. For treatment of migraines after onset (i.e. acute treatment) pain relieving medication is administered. Pain relieving medications for the treatment of migraines include those commonly taken for headaches such as aspirin, ibuprofen and acetaminophen. Other pain relieving medications includes triptans and ergots. One particular medication is known as rizatriptan. Rizatriptan is available as a tablet and is specifically used for the treatment of migraines. Rizatriptan tablets are available under the tradename Maxalt® (Maxalt is a registered trademark of and available from Merck & Co.). However, not all individuals suffering from migraines are able to safely or comfortably administer solid oral dosages.

Thus, there is a need in the art for a liquid rizatriptan composition.

### SUMMARY OF THE INVENTION

The present invention is directed to liquid rizatriptan compositions comprising rizatriptan or a salt thereof and a solvent selected from the group consisting of water, ethanol, propylene glycol, polyethylene glycol 400 and a combination thereof.

The present invention is further directed to liquid rizatriptan compositions comprising: from about 2% to about 15.73% w/w rizatriptan; and
a solvent selected from the group consisting of from about 1% to about 97% w/w water, from about 10% to about 60% w/w ethanol, from about 1% to about 50% w/w propylene glycol, from about 1% to about 50% polyethylene glycol 400 and a combination thereof

The present invention is further directed to a method of treating migraines comprising administering to a subject in need thereof an effective amount of a composition of the present invention.

### BRIEF DESCRIPTION OF THE DRAWINGS

FIG. 1. Pharmacokinetic profiles of rizatriptan benzoate or base in beagle dogs following nasal or oral administration.

### DETAILED DESCRIPTION OF THE INVENTION

The Applicant has surprisingly developed liquid rizatriptan compositions suitable for nasal administration. The rizatriptan compositions are storage stable and unexpectedly more effective than an oral administration of a solid dosage form of rizatriptan.

In one embodiment, the present invention is directed to liquid rizatriptan compositions comprising rizatriptan or a salt thereof and a solvent selected from the group consisting of water, ethanol, propylene glycol, polyethylene glycol 400 and a combination thereof.

Rizatriptan may be in base form or in the form of a salt. Salts that can be used in accordance with the current invention include but are not limited to hydrochloride, hydrochloride dihydrate, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. In a preferred embodiment the salt is benzoate.

Rizatriptan benzoate may be present in the compositions of the invention at a concentration from about 1% to about 20% w/w, preferably from about 2% to about 15.73% w/w, or from about 2.91% to about 15.73% w/w and most preferably at about 7.414% w/w. Equivalent concentrations of rizatriptan base may also be used.

Solvents suitable for use in the present invention include, but are not limited to, water, ethanol, propylene glycol and polyethylene glycol 400 (“PEG-400”). In a preferred embodiment the water is United States Pharmacopeia (“USP”) purified water.

Solvents may be present in the compositions of the invention at a concentration from about 1% to about 99% w/w, preferably from about 1% to about 97% w/w, from about 10% to about 60% w/w and from about 1% to about 50% w/w.

Water may be present in the compositions of the invention at a concentration from about 1% to about 97% w/w, preferably from about 27% to about 76% w/w.

Ethanol may be present in the compositions of the invention at a concentration from about 10% to about 60% w/w, preferably from about 9% to about 50% w/w and more preferably at about 20% w/w.

Propylene glycol may be present in the compositions of the invention at a concentration from about 1% to about 50% w/w, preferably from about 5% to about 43% w/w and more preferably at about 10% w/w.

PEG-400 may be present in the compositions of the invention at a concentration from about 1% to about 50% w/w, preferably from about 8% to about 28% w/w and more preferably at about 15% w/w.

In another embodiment, the compositions of the present invention further comprise one or more agents selected from the group consisting of a solubilizing agent, a stabilizer, a permeation enhancer, a preservative, a pH modifier, a sweetener and a flavoring agent.

Solubilizing agents suitable for use in the present invention include, but are not limited to, polysorbates and inclusion complex forming agents. Polysorbates include, but are not limited to, polysorbate 20, 40, 60 and 80. Polysorbates are sold under the tradename Tween® (Tween is a registered trademark of and available from Croda Americas LLC.) Inclusion complex forming agents are chemical species capable of forming an inclusion complex with rizatriptan. In a preferred embodiment, the inclusion complex forming agents are cyclodextrins. Cyclodextrins suitable for use in the present invention are water-soluble substituted or unsubstituted cyclodextrins or their derivatives thereof which can be well tolerated when administered nasally, which include α, β- or γ-cyclodextrins or derivatives thereof, preferably derivatives wherein one or more of the hydroxy groups are substituted, e.g. by alky, hydroxyalkyl, carboxyalkyl, alkylcarbonyl, carboxyalkoxyakyl, alkyl carbonyloxyalkyl, alkoxycarbonylalkyl or hydroxy-(mono or polyalkoxy)alkyl groups, wherein each alkyl or alkylene moiety preferably contains up to six carbons. Substituted cyclodextrins suitable for use in the present invention, include ethers, polyethers or mixed ethers thereof. Preferably such substituted cyclodextrins are ethers wherein the hydrogen of one or more cyclodextrin hydroxy groups is replaced by one or more cyclodextrin hydroxy groups is replaced by C₁₋₃alkyl, hydroxy-C₂₋₄malkyl or carboxy-C₁₋₂allyl or more particularly by methyl, ethyl, hydroxyethyl, hydroxypropyl, hydroxybutyl, carboxymethyl or carboxyethyl. In a most preferred embodiment, cyclodextrins used in the present invention are beta cyclodextrin ethers and polyethers such as dimethyl beta cyclodextrin, hydroxypropyl beta cyclodextrin, hydroxyethyl-β-cyclodextrin and sulfobutylether beta cyclodextrin. Hydroxypropyl beta cyclodextrins are sold under the tradename Kleptose® (Kleptose is a trademark of and available from Roquette Freres Corporation, France). Sulfobutylethyer beta cyclodextrins are sold under the tradename Captisol® (Captisol is a trademark of and available from Cydex Pharmaceuticals, Inc.).

Solubilizing agents may be present in the compositions of the invention at a concentration from about 1% to about 50% w/w, more preferably from about 10% to about 40% w/w and most preferably about 10% or about 34% w/w.

Stabilizers suitable for use in the present invention include, but are not limited to, D,L-alpha tocopherol, butylated hydroxytoluene, methionine, ascorbyl palmitate, ascorbic acid, butylated hydroxyanisole, citric acid, ethylenediamine tetra acetic acid, sodium bisulfate, tert-butylhydroquinone and propyl gallate.

Stabilizers may be present in the compositions of the invention at a concentration from about 0.002% to about 0.2% w/w, preferably from about 0.05% to about 0.2% w/w, from about 0.001% to about 0.04% w/w, from about 0.01% to about 0.1% w/w, from about 0.005% to about 0.1% w/w or about 0.005% to about 0.03% w/w.

Permeation enhancers suitable for use in the present invention include, but are not limited to, octanoic acid, oleic acid, polysorbate 80, menthol, edetate disodium, sodium edetate, cetylpyridinium chloride, sodium lauryl sulfate, citric acid, sodium desoxycholate, sodium deoxyglycolate, glyceryl oleate and L-lysine.

Permeation enhancers may be present in the compositions of the invention at a concentration from about 0.02% to about 7.8%, preferably from about 0.02% to about 0.5% and more preferably at about 0.5%.

Preservatives suitable for use in the present invention include, but are not limited to, benzalkonium chloride, butyl paraben, methyl paraben, ethyl paraben, propyl paraben, sodium benzoate and benzoic acid.

Preservatives may be present in the compositions of the invention at a concentration from about 0.005% to about 0.2% w/w, preferably from about 0.01% to about 0.03% w/w, from about 0.005% to about 0.03% w/w, from about 0.01% to about 0.05% w/w and from about 0.03% to about 0.2% w/w.

Flavoring agents suitable for use in the present invention include, but are not limited to, raspberry, peppermint oil, grape flavor, menthol, spearmint oil, citrus oil, cinnamon oil, strawberry flavor, cherry flavor, raspberry flavor, orange oil, lemon oil, lemon mint flavor, fruit punch flavor, and combinations thereof. In a preferred embodiment, the formulations contain strawberry flavor.

Flavoring agents may be present in compositions of the invention at a concentration from about 0.001% to about 1% w/w.

Sweeteners suitable for use in the present invention include, but are not limited to, sucralose, sucrose, aspartame, saccharin, dextrose, mannitol, xylitol, and combinations thereof.

Sweeteners may be present in the compositions of the invention at a concentration from about 0.001% to about 1% w/w.

In a preferred embodiment, the pH from about 3 to about 11, more preferably from about 4 to about 6 and most preferably about 5.5. The pH may be modified using a pH modifier. pH modifiers suitable for use in the present invention include, but are not limited to, dilute hydrochloric acid, citric acid, fumaric acid, lactic acid or with dilute sodium hydroxide, sodium citrate, sodium bicarbonate, sodium carbonate, and ammonium carbonate.

In another preferred embodiment, compositions of the present invention provide a spray pattern characteristic selected from the group consisting of a Dmin from about 10 to about 20 millimeters, a Dmax from about 15 to about 30 millimeters, an ovality ratio from about 0.5 to about 3 at 6 centimeters from a spray nozzle, a plume width from about 10 to about 30 millimeters at 3 centimeters from a spray nozzle, a plume width from about 20 to about 40 millimeters at 6 centimeters from a spray nozzle, a plume angle from about 25 to about 45° at 3 centimeters from a spray nozzle and/or a plume angle from about 15 to about 35° at 6 centimeters from a spray nozzle.

In another preferred embodiment, compositions of the present invention provide a particle size parameter at 3 centimeters from a spray nozzle selected from the group consisting of a DV(10) from about 10 to about 30 microns, a DV(50) from about 20 to about 60 microns, a DV(90) from about 60 to about 150 microns and/or a span from about 1 to about 5.5.

In another preferred embodiment, compositions of the present invention provide a particle size parameter at 6 centimeters from a spray nozzle selected from the group consisting of a DV(10) from about 10 to about 40 microns, a DV(50) from about 20 to about 60 microns, a DV(90) from about 50 to about 150 microns and/or a span from about 1 to about 3.5.

In another preferred embodiment, compositions of the present invention provide from about 1 to about 8% of total particles at less than 10 microns when measured at 3 or at 6 centimeters from a spray nozzle.

In another preferred embodiment, the present invention is directed to a liquid rizatriptan composition comprising:
about 14.534% w/w rizatriptan benzoate;
about 50% w/w ethanol;
about 29.346% w/w water;
optionally, about 0.1% w/w vitamin E;
optionally, about 0.02% w/w edetate disodium, dihydrate;
optionally, about 0.5% w/w menthol; and
optionally, about 0.5% w/w sucralose.
   wherein, optionally the composition has pH of about 5.5.

In another preferred embodiment, compositions of the present invention provide provides a spray characteristic selected from the group consisting of a plume width from about 15 to about 30 millimeters at 3 centimeters from a spray nozzle, a plume width from about 20 to about 40 millimeters at 6 centimeters from a spray nozzle, a plume angle from about 20 to about 50° at 3 centimeters from a spray nozzle, a plume angle from about 20 to about 35° at 6 centimeters from a spray nozzle, a Dmin from about 10 to about 25 millimeters, a Dmax from about 15 to about 30 millimeters, an ovality ratio from about 1 to about 2 at 3 centimeters from a spray nozzle, a Dmin from about 20 to about 30 millimeters, a Dmax from about 25 to about 45 millimeters, an ovality ratio from about 1 to about 2 at 6 centimeters from a spray nozzle.

In another preferred embodiment, compositions of the present invention provide provides a particle size parameter selected from the group consisting of: at 3 centimeters from a spray nozzle consisting of a DV(10) from about 15 to about 30 microns, a DV(50) from about 35 to about 55 microns, a DV(90) from about 60 to about 150 microns, a span from about 1 to about 5; and at 6 centimeters from a spray nozzle selected from the group consisting of a DV(10) from about 10 to about 40 microns, a DV(50) from about 20 to about 60 microns, a DV(90) from about 60 to about 150 microns, a span from about 1 to about 5.

In another preferred embodiment, compositions of the present invention provide a Cmax from about 100 to about 650 nanograms per milliliter, a Tmax from about 3 to about 20 minutes, and/or an AUC from 3,500 to about 9,000 nanogram minutes per milliliter following nasal administration to a beagle dog.

In another preferred embodiment, the present invention is directed to a method of treating migraines comprising administering to a subject in need thereof an effective amount of a composition of the present invention.

In another preferred embodiment, the compositions of the present invention are administered nasally via a spray pump at an amount from about 50 to about 200 microliters.

### Definitions

As used herein, all numerical values relating to amounts, weights, and the like, that are defined as “about” each particular value is plus or minus 10%. For example, the phrase “about 10% w/w” is to be understood as “9% to 11% w/w.” Therefore, amounts within 10% of the claimed value are encompassed by the scope of the claims.

As used herein “% w/w” refers to the percent weight of the total formulation.

As used herein the term “effective amount” refers to the amount necessary to treat a patient in need thereof.

As used herein the term “treatment” or “treating” refers to reversing, alleviating, inhibiting, or slowing the progress of the disease, disorder, or condition to which such terms apply, or one or more symptoms of such disease, disorder, or condition.

As used herein the term “subject” refers but is not limited to a person that suffers from migraines.

As used herein the term “migraines” refers to a chronic and/or episodic condition including moderate to severe pulsating unilateral headaches lasting between 4 and 72 h, which includes migraine without aura and migraine with aura.

As used herein, the phrase “migraine without aura” refers to at least five attacks fulfilling the following criteria: (a) the headache attack lasts 4-72 hours with the headache having at least two of the following features: unilateral location, pulsating quality, moderate or severe intensity with direct influence on activities of daily living, and aggravation by daily activities; and (b) during the headache at least one of the following occurs: nausea and/or vomiting, and photophobia and phonophobia.

As used herein, “migraine with aura” refers to at least two attacks accompanied by at least 3 of the 4 following features: (a) one or more fully reversible aura symptoms; (b) at least one aura symptom which develops gradually over more than four minutes or two or more symptoms which occur in succession; (c) no aura symptom which lasts more than 60 minutes; (d) a headache occurs prior to, simultaneously with or following the aura, with a free interval between aura and headache of less than about 60 minutes.

As used herein, the term “aura” refers to a perceptual disturbance experienced before a headache begins. Perceptual disturbances include, but are not limited to, perception of a strange light including flashing lights or blind spots, an unpleasant smell, confusing thoughts or experiences or tingling on one side of the face or one extremity.

As used herein the phrase “pharmaceutically acceptable” refers to ingredients that are not biologically or otherwise undesirable in a sublingual or intranasal dosage form.

As used herein, the term “stable” includes but is not limited physical and chemical stability.

Preferably, the compositions of the present invention are propellant free. As used herein, “propellant free” refers to a formulation that is not administered using compressed gas.

The disclosed embodiments are simply exemplary embodiments of the inventive concepts disclosed herein and should not be considered as limiting, unless the claims expressly state otherwise.

The following examples are intended to illustrate the present invention and to teach one of ordinary skill in the art how to use the formulations of the invention. They are not intended to be limiting in any way.

### EXAMPLES

### Example 1

### Preparation of Compositions of the Invention

TABLE 1
Rizatriptan Compositions

| | Composition | | | | | | | |
|---|---|---|---|---|---|---|---|---|
| | #1 | #2 | #3 | #4 | #5 | #6 | #7 | #8 |
| Rizatriptan benzoate | 15.73 | 15.73 | 11.874 | 15.73 | 5.45 | 14.534 | 14.534 | 14.534 |
| Dehydrated alcohol | 50 | 50 | 50 | 40 | 50 | — | 50 | 50 |
| Propylene glycol | 5 | — | — | 5 | — | — | 5 | 5 |
| Octanoic acid | — | 2 | 7.8 | 5.4 | 2 | — | — | — |
| Glycerin | — | — | — | — | 30 | — | — | — |
| Benzalkonium chloride | — | 0.02 | — | — | — | — | — | — |
| Menthol | 0.5 | 0.5 | 0.5 | 0.5 | 0.5 | — | — | — |
| Cetylpyridinium chloride | 0.5 | — | — | 1 | — | — | — | — |
| Sucralose | 0.5 | 0.5 | 0.5 | 0.5 | 0.5 | — | — | — |
| Strawberry Flavor | 0.2 | 0.2 | 0.2 | 0.2 | 0.2 | — | — | — |
| Vitamin E | 0.1 | 0.1 | 0.1 | 0.1 | 0.1 | — | 0.1 | 0.1 |
| Edetate Disodium | 0.02 | 0.02 | 0.02 | 0.02 | 0.02 | 0.02 | 0.02 | 0.02 |
| Ascorbyl palmitate | — | — | — | — | — | — | — | 0.05 |
| Ascorbic acid | — | — | — | — | — | — | 0.05 | — |
| Sodium citrate anhydrate | — | — | — | — | — | 0.05 | — | — |
| Tween ® 80 | — | — | — | — | — | 10 | — | — |
| Water, USP | 27.45 | 30.93 | 29.006 | 31.55 | 11.23 | 75.396 | 30.296 | 30.296 |
| pH | 5.5 | 5.5 | 5.5 | 5.5 | 5.5 | 9.8 | 6 | 6 |

TABLE 2
Additional Rizatriptan Compositions

| | Composition | | | | | |
|---|---|---|---|---|---|---|
| | #9 | #10 | #11 | #12 | #13 | #14 |
| Rizatriptan benzoate | 14.534 | 14.534 | 14.534 | 14.534 | 7.265 | 7.265 |
| Dehydrated alcohol | 50 | 50 | 17 | 25.6 | 9.3 | 9.3 |
| Propylene glycol | 5 | 5 | 17 | — | 27.8 | — |
| PEG-400 | — | — | — | 8.5 | — | 27.8 |
| Butylated hydroxytoluene | — | — | — | — | — | — |
| Butylated | — | — | — | — | — | — |
| hydroxyanisole | | | | | | |
| tert-Butylhydroquinone | — | — | — | — | — | — |
| Edetate disodium | — | — | — | — | — | — |
| Benzalkonium chloride | — | — | — | — | — | — |
| Methionine | 0.05 | — | — | — | — | — |
| Sodium bisulfite | — | 0.1 | — | — | — | — |
| Captisol ® | — | — | — | — | — | — |
| Kleptose ® | — | — | — | — | — | — |
| Water, USP | 30.416 | 30.366 | 51.28 | 51.28 | 55.641 | 55.641 |
| pH | 5 | 7.5 | 5 | 5 | 5 | 5 |
| | Composition | | | | | |
| | #15 | #16 | #17 | #18 | #19 | #20 |
| Rizatriptan benzoate | 7.265 | 7.265 | 7.265 | 7.265 | — | 7.3 |
| Rizatriptan base | — | — | — | — | 5 | — |
| Dehydrated alcohol | — | — | — | — | 20 | — |
| Propylene glycol | — | — | 43 | — | — | 10 |
| PEG-400 | — | — | — | 22 | — | 15 |
| Butylated hydroxytoluene | — | — | — | — | 0.002 | |
| Butylated | — | — | — | — | 0.02 | 0.01 |
| hydroxyanisole | | | | | | |
| tert-Butylhydroquinone | — | — | — | — | — | 0.005 |
| Edetate disodium | — | — | — | — | 0.05 | 0.05 |
| Benzalkonium chloride | — | — | — | — | 0.02 | 0.02 |
| Methionine | — | — | — | — | — | — |
| Sodium bisulfite | — | — | — | — | — | — |
| Captisol ® | 34 | — | — | — | — | — |
| Kleptose ® | — | 34 | — | — | — | — |
| Water, USP | 58.735 | 58.735 | 49.735 | 70.735 | 74.908 | 67.615 |
| pH | 5.5 | 5.6 | 5.6 | 5.6 | 5.5 | 5.5 |

TABLE 3
Additional Rizatriptan Compositions

| | Composition | | | | |
|---|---|---|---|---|---|
| | #21 | #22 | #23 | #24 | #25 |
| Rizatriptan benzoate | 14.534 | 7.3 | 7.3 | 7.3 | 7.414 |
| Alcohol | 50 | 20 | — | | 20 |
| Propylene glycol | 5 | — | 10 | 10 | — |
| PEG-400 | — | — | 15 | 15 | — |
| D,L α-tocopherol | 0.1 | — | — | — | — |
| Butylated hydroxytoluene | — | 0.002 | — | — | 0.002 |
| Butylated hydroxyanisole | — | 0.02 | 0.01 | 0.01 | 0.02 |
| Edetate disodium | 0.02 | 0.05 | 0.05 | 0.05 | 0.05 |
| Propyl gallate | — | — | — | 0.005 | — |
| Benzalkonium Chloride | — | 0.02 | 0.02 | 0.02 | 0.02 |
| Sucralose | — | — | — | — | 0.1 |
| Water, USP | QS | QS | QS | QS | QS |
| pH | 5.5 | 5.5 | 5.5 | 5.5 | 5.5 |

Excipients listed in Table 1-3, above, were dissolved in either the ethanol or purified water based on their solubility. The solutions were mixed together and rizatriptan benzoate or rizatriptan base (active pharmaceutical ingredient) was added to the final solution and mixed until dissolved. The pH of the resulting solution was adjusted using HCl or NaOH of appropriate strength.

### Example 2

### Stability of Compositions of the Invention

### Method

Compositions 1-24, listed in Tables 1-3, above, were subjected to stability testing at 55° C., 40±2° C./75±5% relative humidity (“RH”) and/or 25±2° C./60±5% RH to identify suitable stabilizer or combination of stabilizers. Stability samples were collected at predetermined timepoints. Assay and impurities were quantified using high performance liquid chromatography with an ultraviolet detector. The assay was performed at 280 nm and indicated as a % of initial concentration. For all impurities, analysis was performed at 280 nm and expressed as a % area of assay.

TABLE 4
Stability data for compositions #1-#20 stored at 55° C.

| Composition | | RRT | T = 0 | 1 week | 6 Week | 8 Week |
|---|---|---|---|---|---|---|
| #1 | Assay | 1.000 | 100% | — | — | 98.32% |
| | Unknown Impurity | 1.235 | ND | — | — | 0.13% |
| | Total Impurities | — | 0.08% | — | — | 0.43% |
| #2 | Assay | 1.000 | 100% | — | — | 99.22% |
| | Unknown Impurity | 1.235 | ND | — | — | 0.14% |
| | Total Impurities | — | 0.08% | — | — | 0.50% |
| #3 | Assay | 1.000 | 100% | — | — | 102.02% |
| | Unknown Impurity | 1.227 | ND | — | — | 0.14% |
| | Total Impurities | — | 0.08% | — | — | 0.44% |
| #4 | Assay | 1.000 | 100% | 100.73% | 99.88% | 99.05% |
| | Impurity H | 1.490 | ND | 0.01% | 0.06% | 0.08% |
| | Unknown Impurity | 1.255 | 0.01% | 0.01% | 0.15% | 0.18% |
| | Total Impurities | — | 0.10% | 0.13% | 0.57% | 0.70% |
| #5 | Assay | 1.000 | 100% | 99.15% | — | — |
| | Impurity I | 0.888 | 0.02% | 0.02% | — | — |
| | Impurity H | 1.450 | ND | 0.02% | — | — |
| | Impurity A | 2.243 | ND | 0.01% | — | — |
| | Total Impurities | — | 0.06% | 0.08% | — | — |
| #6 | Assay | 1.000 | 100% | 98.32% | — | — |
| | Impurity I | 0.916 | 0.02% | 0.16% | — | — |
| | Impurity H | 1.355 | 0.01% | 0.12% | — | — |
| | Unknown Impurity | 0.887 | 0.01% | 0.26% | — | — |
| | Total Impurities | — | 0.10% | 0.68% | — | — |
| #7 | Assay | 1.000 | 100% | — | 97.86% | 97.64% |
| | Impurity H | 1.351 | ND | — | 0.15% | 0.21% |
| | Unknown Impurity | 0.559 | ND | — | 0.07% | 0.11% |
| | | 0.885 | 0.01% | — | 0.11% | 0.17% |
| | | 1.237 | ND | — | 0.31% | 0.36% |
| | | 1.919 | ND | — | 0.12% | 0.16% |
| | | 3.031 | ND | — | 0.04% | 0.05% |
| | Total Impurities | — | 0.13% | — | 1.02% | 1.33% |
| #8 | Assay | 1.000 | 100% | — | 97.59% | 96.83% |
| | Impurity H | 1.351 | ND | — | 0.12% | 0.16% |
| | Unknown Impurity | 0.887 | 0.01% | — | 0.10% | 0.16% |
| | | 1.237 | ND | — | 0.23% | 0.27% |
| | | 1.918 | ND | — | 0.09% | 0.13% |
| | Total Impurities | — | 0.10% | — | 0.81% | 1.15% |
| #9 | Assay | 1.000 | 100% | 100.28% | — | 93.73% |
| | Unknown Impurity | 0.472 | ND | 0.01% | — | 0.20% |
| | | 0.549 | ND | 0.02% | — | 0.28% |
| | | 0.898 | ND | 0.02% | — | 0.20% |
| | | 1.937 | ND | 0.05% | — | 0.44% |
| | | 2.709 | ND | ND | — | 0.20% |
| | | 2.937 | ND | 0.10% | — | 0.64% |
| | Total Impurities | — | 0.09% | 0.30% | — | 2.78% |
| #10 | Assay | 1.000 | 100% | 99.47% | — | — |
| | Impurity I | 0.928 | 0.03% | 0.19% | — | — |
| | Unknown Impurity | 0.549 | 0.18% | 0.20% | — | — |
| | | 0.898 | ND | 0.17% | — | — |
| | Total Impurities | — | 0.66% | 0.92% | — | — |
| #11 | Assay | 1.000 | 100.00% | 99.12% | — | — |
| | Unknown Impurity | 0.527 | 0.01% | 0.10% | — | — |
| | | 0.891 | ND | 0.14% | — | — |
| | | 1.942 | ND | 0.14% | — | — |
| | | 2.996 | ND | 0.27% | — | — |
| | Total Impurities | — | 0.11% | 0.92% | — | — |
| #12 | Assay | 1.000 | 100% | 99.70% | — | — |
| | Unknown Impurity | 0.890 | 0.01% | 0.24% | — | — |
| | | 1.940 | ND | 0.20% | — | — |
| | | 2.984 | ND | 0.43% | — | — |
| | Total Impurities | — | 0.12% | 1.32% | — | — |
| #13 | Assay | 1.000 | 100% | 100.36% | — | — |
| | Unknown Impurity | 0.889 | 0.01% | 0.14% | — | — |
| | | 1.943 | ND | 0.14% | — | — |
| | | 2.995 | ND | 0.32% | — | — |
| | Total Impurities | — | 0.09% | 0.87% | — | — |
| #14 | Assay (% of Initial Conc.) | 1.000 | 100% | 99.31% | — | — |
| | Unknown Impurity | 0.556 | 0.02% | 0.15% | — | — |
| | | 0.891 | 0.01% | 0.26% | — | — |
| | | 1.919 | 0.01% | 0.30% | — | — |
| | | 2.749 | ND | 0.11% | — | — |
| | | 2.990 | 0.01% | 0.56% | — | — |
| | Total Impurities | — | 0.17% | 1.72% | — | — |
| #15 | Assay | 1.000 | 100% | — | 96.2 | 96.25 |
| | Impurity I | 0.887 | 0.01% | — | 0.71% | 0.80% |
| | Unknown Impurity | 0.477 | ND | — | 0.14% | 0.17% |
| | | 0.553 | ND | — | 0.20% | 0.23% |
| | | 1.935 | ND | — | 0.54% | 0.60% |
| | | 2.690 | ND | — | 0.64% | 0.78% |
| | | 2.962 | 0.01% | — | 1.21% | 1.25% |
| | | 3.120 | ND | — | 0.10% | 0.11% |
| | Total Impurities | — | 0.10% | — | 3.86% | 4.49% |
| #16 | Assay | 1.000 | 100 | — | 97.76 | 97.65 |
| | Impurity I | 0.887 | 0.01% | — | 0.54% | 0.67% |
| | Unknown Impurity | 0.477 | ND | — | 0.08% | 0.11% |
| | | 0.553 | ND | — | 0.15% | 0.19% |
| | | 1.935 | ND | — | 0.34% | 0.41% |
| | | 2.690 | ND | — | 0.30% | 0.43% |
| | | 2.930 | ND | — | 0.86% | 0.97% |
| | Total Impurities | — | 0.10% | — | 2.60% | 3.29% |
| #17 | Assay | 1.000 | 100% | 100.55% | — | — |
| | Impurity C | 1.262 | BQL | BQL | — | — |
| | Unknown Impurity | 1.959 | ND | 0.06% | — | — |
| | | 3.018 | ND | 0.19% | — | — |
| | Total Impurities | — | 0.08% | 0.25% | — | — |
| #18 | Assay | 1.000 | 100% | 99.77% | — | — |
| | Unknown Impurity | 0.485 | ND | 0.05% | — | — |
| | | 0.892 | BQL | 0.30% | — | — |
| | | 1.960 | ND | 0.21% | — | — |
| | | 2.777 | BQL | 0.14% | — | — |
| | | 3.020 | ND | 0.79% | — | — |
| | Total Impurities | — | 0.08% | 1.49% | — | — |
| #19 | Assay | 1.000 | 100% | 99.09% | — | 100.54% |
| | Unknown Impurity | 1.087 | ND | 0.07% | — | 0.18% |
| | Total Impurities | — | 0.12 | 0.22% | — | 0.79% |
| #20 | Assay | 1.000 | 100% | 99.27% | — | 97.59% |
| | Unknown Impurity | 1.490 | ND | 0.03% | — | 0.10% |
| | | 0.407 | ND | 0.02% | — | 0.16% |
| | | 0.484 | ND | 0.05% | — | 0.20% |
| | | 0.852 | ND | 0.06% | — | 0.30% |
| | | 2.562 | ND | 0.02% | — | 0.13% |
| | Total Impurities | — | 0.06% | 0.29% | — | 1.19% |

ND = Not detected
BQL = Below quantifiable levels

TABLE 5
Stability data for composition #21 stored at 55° C.,
40 ± 2° C./75 ± 5% RH and at 25 ± 2° C./60 ± 5% RH

| | | | | | 40 ± 2° C./ | | 25 ± 2° C./ | | |
|---|---|---|---|---|---|---|---|---|---|
| Composition #21 | | | 55° C. | | 75 ± 5% RH | | 60 ± 5% RH | | |
| | RRT | T0 | 4 week | 8 week | 3 month | 6 month | 3 month | 6 month | 12 month |
| Assay | 1 | 100% | 97.91% | 98.45% | 98.90% | 97.98% | 98.76% | 98.74% | 99.49% |
| Impurity I | 0.916 | 0.02% | 0.02% | 0.03% | 0.02% | 0.01% | 0.01% | 0.01% | 0.01% |
| Impurity C | 1.330 | 0.01% | ND | ND | ND | ND | 0.01% | 0.01% | 0.01% |
| Impurity H | 1.352 | ND | 0.04% | 0.08% | 0.02% | 0.05% | ND | 0.01% | 0.01% |
| Unknown impurity | 1.237 | ND | 0.06% | 0.14% | 0.02% | 0.01% | ND | 0.01% | 0.01% |
| Total Impurities | — | 0.09% | 0.30% | 0.64% | 0.16% | 0.29% | 0.08% | 0.08% | 0.13% |

ND = Not detected

TABLE 6
Stability data for composition #22 stored at 55° C.,
40 ± 2° C./75 ± 5% RH and at 25 ± 2° C./60 ± 5% RH

| | | | | | 40 ± 2° C./ | | 25 ± 2° C./ | |
|---|---|---|---|---|---|---|---|---|
| Composition #22 | | | 55° C. | | 75 ± 5% RH | | 60 ± 5% RH | |
| | RRT | T0 | 4 week | 8 week | 3 month | 6 month | 3 month | 6 month |
| Assay | 1 | 100.00% | 100.81% | 101.42% | 101.16% | 100.85% | 100.83% | 100.51% |
| Impurity I | 0.901 | 0.01% | 0.02% | 0.02% | 0.01% | 0.01% | 0.01% | 0.01% |
| Impurity H | 1.490 | ND | 0.03% | 0.05% | 0.01% | 0.03% | ND | ND |
| Impurity A | 2.238 | ND | ND | 0.01% | ND | ND | ND | ND |
| Unknown impurity | 0.859 | ND | 0.05% | 0.11% | 0.02% | 0.04% | ND | ND |
| Total Impurities | — | 0.08% | 0.31% | 0.50% | 0.18% | 0.33% | 0.09% | 0.08% |

ND = Not detected

TABLE 7
Stability data for rizatriptan benzoate composition #23 stored at 55° C.,
40 ± 2° C./75 ± 5% RH and at 25 ± 2° C./60 ± 5% RH.

| | | | | | 40 ± 2° C./ | | 25 ± 2° C./ | |
|---|---|---|---|---|---|---|---|---|
| Composition #23 | | | 55° C. | | 75 ± 5% RH | | 60 ± 5% RH | |
| | RRT | T0 | 4 week | 8 week | 3 month | 6 month | 3 month | 6 month |
| Assay | 1 | 100.00% | 101.19% | 99.41% | 99.68% | 99.81% | 100.30% | 99.96% |
| Impurity I | 0.901 | 0.01% | 0.01% | 0.01% | ND | ND | 0.01% | 0.01% |
| Impurity H | 1.490 | 0.01% | 0.05% | 0.08% | 0.03% | 0.05% | 0.02% | 0.02% |
| Unknown | 0.417 | ND | 0.05% | 0.10% | 0.03% | ND | ND | ND |
| impurity | 0.494 | ND | 0.06% | 0.12% | 0.03% | 0.07% | 0.01% | 0.01% |
| | 0.859 | ND | 0.18% | 0.27% | 0.11% | 0.16% | 0.04% | 0.06% |
| | 2.520 | ND | 0.06% | 0.11% | 0.01% | ND | ND | ND |
| Total Impurities | — | 0.12% | 0.59% | 0.99% | 0.34% | 0.56% | 0.14% | 0.22% |

ND = Not detected

TABLE 8
Stability data for composition #24 stored at 55° C., 40 ± 2° C./75 ± 5% RH and at
25 ± 2° C./60 ± 5% RH

| | | | | | 40 ± 2° C./ | | 25 ± 2° C./ | |
|---|---|---|---|---|---|---|---|---|
| Composition #24 | | | 55° C. | | 75 ± 5% RH | | 60 ± 5% RH | |
| | RRT | T0 | 4 week | 8 week | 3 month | 6 month | 3 month | 6 month |
| Assay | 1 | 100.00% | 100.30% | 99.91% | 99.31% | 99.14% | 98.18% | 98.93% |
| Impurity I | 0.901 | 0.02% | ND | 0.01% | 0.01% | ND | 0.01% | 0.01% |
| Impurity H | 1.490 | ND | 0.03% | 0.06% | 0.02% | 0.03% | 0.01% | 0.01% |
| Impurity A | 2.309 | ND | ND | ND | ND | ND | ND | ND |
| Unknown impurity | 0.852 | ND | 0.12% | 0.20% | 0.09% | 0.11% | 0.03% | 0.04% |
| Total Impurities | — | 0.07% | 0.31% | 0.61% | 0.23% | 0.34% | 0.10% | 0.15% |

ND = Not detected

### Result

Compositions #1-#4, #19 and #21-#24 all had total impurities levels below 1% after 8 weeks at 55° C.

### Example 3

### Pharmacokinetics of Rizatriptan Benzoate or Base Compositions in Beagle Dogs

TABLE 9
Additional Rizatriptan compositions

| | Composition | | | | | |
|---|---|---|---|---|---|---|
| | #26 | #27 | #28 | #29 | #30 | #31 |
| Rizatriptan Benzoate | 2.963 | 2.907 | — | 2.91 | 2.94 | 2.92 |
| Rizatriptan base | — | — | 2 | — | — | — |
| Dehydrated alcohol | 20 | — | — | — | 10 | — |
| Propylene glycol | — | 10 | — | — | 5 | — |
| PEG-400 | — | 15 | — | — | | — |
| Butylated | 0.002 | — | — | — | — | — |
| hydroxytoluene | | | | | | |
| Butylated | 0.02 | 0.01 | — | — | 0.01 | — |
| hydroxyanisole | | | | | | |
| Edetate disodium | 0.05 | 0.05 | 0.05 | 0.05 | 0.05 | — |
| Methylparaben sodium | — | 0.03 | — | — | 0.03 | — |
| Propylparaben sodium | — | 0.02 | — | — | 0.02 | — |
| Benzalkonium chloride | 0.02 | — | 0.02 | — | — | — |
| Anhydrous citric acid | — | — | — | 0.043 | — | — |
| Water, USP | 76.945 | 71.983 | 97.93 | 96.997 | 81.95 | — |
| Capsule | — | — | — | — | — | Yes |
| Route of | Nasal | Nasal | Nasal | Nasal | Nasal | Oral |
| administration | | | | | | |

### Methods

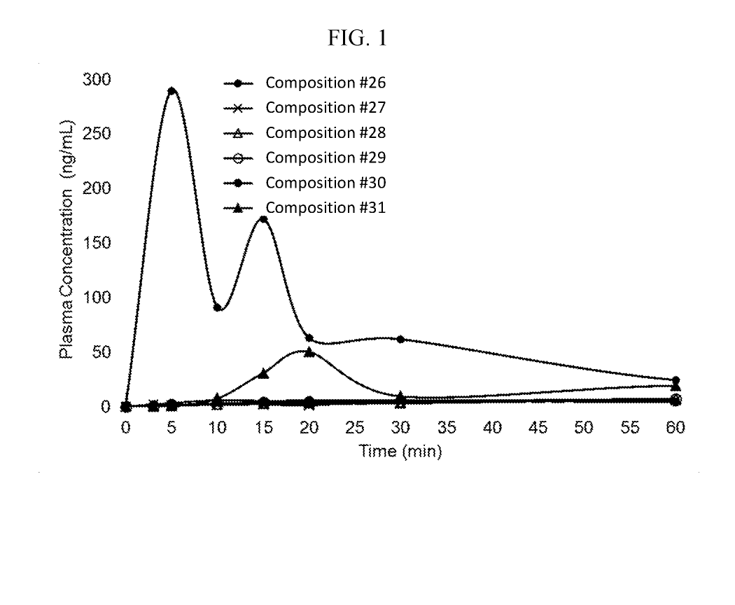

In a single dose, five healthy male beagle dogs weighing approximately 10 kilograms each were nasally administered rizatriptan benzoate or base compositions #26 to #30 from Table 9, above. Composition #31 was rizatriptan benzoate in capsule, which was administered orally. The dogs were fasted overnight till four hours post administration. Each dosing was followed by a one-week washout period. Blood samples were taken prior to administration and 3, 5, 10, 15, 20, 30 min, 1, 1.5, 2, 4, 8 and 24 hours post dose. Plasma samples were measured for rizatriptan concentration via liquid chromatography-tandem mass spectrometry. Peak concentration in plasma (Cₘₐₓ) and area under the concentration-time curve post-dose (AUC_{0-24 h}) were calculated. Results of this assay can be seen in FIG. 1 and Table 10, below.

TABLE 10
Summary of pharmacokinetic parameters of rizatriptan benzoate or
base in beagle dogs following nasal or oral administration.

| | | | Median | Geo-mean | Geo-mean |
|---|---|---|---|---|---|
| | | | Tmax | Cmax | AUC (0-24 hr) |
| Composition | Dose (mg) | N | (min) | (ng/mL) | (ng * min/mL) |
| #26 | 2 | 5 | 5 | 312 | 8021 |
| #27 | 2 | 5 | 60 | 10 | 1534 |
| #28 | 2 | 5 | 60 | 16 | 1994 |
| #29 | 2 | 5 | 60 | 13 | 1506 |
| #30 | 2 | 5 | 20 | 13 | 1805 |
| #31 | 2 | 5 | 20 | 43 | 3748 |

### Results

Composition #26 had the highest Cmax and AUC_{0-24 h} and shortest Tmax.

### Example 4

### Rizatriptan Benzoate Spray Characterization

Rizatriptan benzoate composition #25 and composition #32 were evaluated for spray characteristics including spray pattern, particle size and plume geometry. See Tables 11-20, below.

TABLE 11
Spray characteristics of composition #25, Spray Pattern at 3 cm and 6 cm

| | Distance 3 cm | | | Distance 6 cm | | |
|---|---|---|---|---|---|---|
| Composition | Dmin | Dmax | Ovality | Dmin | Dmax | Ovality |
| #25 | (mm) | (mm) | Ratio | (mm) | (mm) | Ratio |
| Actuation 1 | 17.23 | 24.68 | 1.432 | 22.16 | 24.49 | 1.105 |
| Actuation 2 | 16.09 | 20.46 | 1.272 | 23.72 | 30.00 | 1.265 |
| Actuation 3 | 15.84 | 18.19 | 1.148 | 22.49 | 37.35 | 1.660 |
| Actuation 4 | 15.86 | 19.81 | 1.250 | 21.15 | 27.82 | 1.316 |
| Actuation 5 | 16.37 | 18.31 | 1.118 | 21.69 | 28.57 | 1.317 |
| Actuation 6 | 16.10 | 20.73 | 1.287 | 21.44 | 28.48 | 1.329 |
| Actuation 7 | 16.28 | 19.25 | 1.183 | 22.18 | 25.44 | 1.147 |
| Actuation 8 | 17.38 | 22.63 | 1.302 | 20.64 | 31.98 | 1.550 |
| Actuation 9 | 15.52 | 19.54 | 1.260 | 22.19 | 34.54 | 1.556 |
| Actuation 10 | 15.88 | 19.89 | 1.252 | 25.76 | 37.15 | 1.442 |
| Average | 16.26 | 20.35 | 1.250 | 22.34 | 30.58 | 1.369 |

TABLE 12
Spray characteristics of composition #25, Particle Size at 3 cm

| | Distance 3 cm | | | | |
|---|---|---|---|---|---|
| Composition | DV (10), | DV (50), | | | |
| #25 | μm | μm | DV (90), μm | <10 μm (%) | Span |
| Actuation 1 | 15.18 | 33.92 | 71.24 | 4.207 | 1.653 |
| Actuation 2 | 14.94 | 32.71 | 68.20 | 4.04 | 1.628 |
| Actuation 3 | 13.14 | 33.10 | 73.75 | 6.032 | 1.831 |
| Actuation 4 | 13.24 | 32.15 | 71.40 | 5.766 | 1.809 |
| Actuation 5 | 13.66 | 32.31 | 70.58 | 5.288 | 1.762 |
| Average | 14.03 | 32.84 | 71.03 | 5.067 | 1.737 |

TABLE 13
Spray characteristics of composition #25, Particle Size at 6 cm

| | Distance 6 cm | | | | |
|---|---|---|---|---|---|
| Composition | DV (10), | DV (50), | | | |
| #25 | μm | μm | DV (90), μm | <10 μm (%) | Span |
| Actuation 1 | 15.97 | 34.85 | 64.66 | 4.791 | 1.397 |
| Actuation 2 | 17.55 | 35.91 | 64.91 | 4.268 | 1.319 |
| Actuation 3 | 17.65 | 35.90 | 64.54 | 4.144 | 1.306 |
| Actuation 4 | 15.99 | 34.11 | 62.62 | 4.761 | 1.367 |
| Actuation 5 | 16.81 | 34.63 | 62.23 | 4.573 | 1.312 |
| Average | 16.79 | 35.08 | 63.79 | 4.507 | 1.340 |

TABLE 14
Spray characteristics of composition #25, Plume geometry data
at 3 cm and 6 cm

| | Distance 3 cm | | Distance 6 cm | |
|---|---|---|---|---|
| Composition #25 | Width (mm) | Angle (°) | Width (mm) | Angle (°) |
| Actuation 1 | 20.80 | 38.0 | 28.43 | 26.6 |
| Actuation 2 | 19.07 | 35.3 | 29.82 | 27.9 |
| Actuation 3 | 18.37 | 34.1 | 27.04 | 25.4 |
| Average | 19.41 | 35.8 | 28.43 | 26.6 |

TABLE 15
Rizatriptan composition # 32

| | Composition |
|---|---|
| | #32 |
| Rizatriptan benzoate | 14.534 |
| Alcohol | 50 |
| Propylene glycol | 5 |
| D,L α-tocopherol | 0.1 |
| Edetate disodium, | 0.02 |
| Dihydrate | |
| Menthol | 0.5 |
| Sucralose | 0.5 |
| Water, USP | 29.346 |
| pH | 5.5 |

Composition #32 was filled in unit dose spray and placed in Freeze/Thaw chamber and Temperature cycling study was performed. Study was consisted of 12-hour cycles, with temperature ranging between freezer temperature −20° C. and 40° C. for a period of 1 month. Composition#32 was evaluated for Assay/Impurity, Spray characterization and Spray content uniformity.

TABLE 16
Assay and impurity of composition #32, Temperature cycling study

| | RRT | T0 | 15 Days | 30 Days |
|---|---|---|---|---|
| Assay | 1 | 100% | 99.42% | 99.80% |
| Impurity I | 0.916 | 0.01 | 0.01 | 0.09 |
| Impurity A | 2.498 | ND | 0.01 | ND |
| Unknown impurity | 2.860 | 0.02 | 0.02 | ND |
| Unknown impurity | 2.948 | 0.01 | 0.01 | ND |
| Total Impurities | — | 0.05 | 0.05 | 0.09 |

TABLE 17
Spray characteristics of composition #32 plume
geometry data at 3 cm and 6 cm

| | Distance 3 cm | | Distance 6 cm | |
|---|---|---|---|---|
| | Width | | | Angle |
| | (mm) | Angle (°) | Width (mm) | (°) |
| Composition #32 T0 | | | | |
| Actuation 1 | 21.64 | 39.70 | 27.58 | 25.90 |
| Actuation 2 | 19.90 | 36.60 | 25.48 | 24.00 |
| Actuation 3 | 21.29 | 39.10 | 26.88 | 25.20 |
| Average | 20.94 | 38.47 | 26.65 | 25.03 |
| Composition #32 T-15 days | | | | |
| Actuation 1 | 23.40 | 42.50 | 32.70 | 30.50 |
| Actuation 2 | 24.44 | 44.30 | 30.29 | 28.30 |
| Actuation 3 | 19.96 | 36.70 | 25.12 | 23.60 |
| Average | 22.60 | 41.17 | 29.37 | 27.47 |
| Composition #32 T-30 days | | | | |
| Actuation 1 | 21.78 | 39.90 | 29.39 | 27.50 |
| Actuation 2 | 21.78 | 39.90 | 27.31 | 25.60 |
| Actuation 3 | 22.47 | 41.00 | 29.39 | 27.50 |
| Average | 22.01 | 40.27 | 28.70 | 26.87 |

TABLE 18
Spray characteristics of composition #32, Spray pattern
data at 3 cm and 6 cm

| | Distance 3 cm | | | Distance 6 cm | | |
|---|---|---|---|---|---|---|
| | Dmin | Dmax | Ovality | Dmin | Dmax | Ovality |
| | (mm) | (mm) | Ratio | (mm) | (mm) | Ratio |
| Composition #32 | | | | | | |
| T0 | | | | | | |
| Actuation 1 | 18.47 | 24.64 | 1.334 | 25.59 | 34.49 | 1.348 |
| Actuation 2 | 18.95 | 24.40 | 1.288 | 23.31 | 36.75 | 1.576 |
| Actuation 3 | 17.33 | 27.18 | 1.568 | 24.27 | 36.61 | 1.508 |
| Average | 18.25 | 25.41 | 1.397 | 24.39 | 35.95 | 1.477 |
| Composition #32 | | | | | | |
| T-15 days | | | | | | |
| Actuation 1 | 20.15 | 26.47 | 1.314 | 25.39 | 36.10 | 1.422 |
| Actuation 2 | 20.17 | 25.82 | 1.280 | 26.81 | 34.33 | 1.281 |
| Actuation 3 | 17.40 | 27.80 | 1.598 | 25.88 | 37.03 | 1.431 |
| Average | 19.24 | 26.70 | 1.397 | 26.03 | 35.82 | 1.378 |
| Composition #32 | | | | | | |
| T-30 days | | | | | | |
| Actuation 1 | 17.70 | 26.14 | 1.477 | 24.32 | 33.67 | 1.385 |
| Actuation 2 | 18.06 | 24.78 | 1.372 | 25.69 | 34.23 | 1.332 |
| Actuation 3 | 18.83 | 24.86 | 1.320 | 24.36 | 31.42 | 1.290 |
| Average | 18.20 | 25.26 | 1.390 | 24.79 | 33.11 | 1.336 |

TABLE 19
Spray characteristics of composition #32, Droplet size Distribution at 3 cm and 6 cm

| | Distance 3 cm | | | | Distance 6 cm | | | |
|---|---|---|---|---|---|---|---|---|
| | DV (10), | | DV (90), | | DV (10), | DV (50), | DV (90), | |
| | μm | DV(50), μm | μm | Span | μm | μm | μm | Span |
| Composition | | | | | | | | |
| #32 | | | | | | | | |
| T0 | | | | | | | | |
| Actuation 1 | 22.79 | 44.85 | 84.40 | 1.374 | 24.74 | 43.75 | 74.86 | 1.146 |
| Actuation 2 | 24.74 | 44.67 | 77.77 | 1.187 | 25.71 | 48.35 | 87.27 | 1.273 |
| Actuation 3 | 21.89 | 42.81 | 79.33 | 1.342 | 23.66 | 45.98 | 84.82 | 1.330 |
| Average | 23.14 | 44.11 | 80.50 | 1.301 | 24.70 | 46.03 | 82.32 | 1.250 |
| Composition | | | | | | | | |
| #32 | | | | | | | | |
| T-15 Days | | | | | | | | |
| Actuation 1 | 23.97 | 47.96 | 95.36 | 1.488 | 28.95 | 52.29 | 90.03 | 1.168 |
| Actuation 2 | 22.40 | 42.54 | 77.34 | 1.291 | 25.72 | 50.39 | 92.50 | 1.325 |
| Actuation 3 | 23.05 | 45.69 | 87.65 | 1.414 | 27.88 | 52.95 | 96.60 | 1.298 |
| Average | 23.14 | 45.40 | 86.78 | 1.398 | 27.52 | 51.88 | 93.04 | 1.264 |
| Composition | | | | | | | | |
| #32 | | | | | | | | |
| T-30 days | | | | | | | | |
| Actuation 1 | 24.24 | 46.33 | 88.45 | 1.386 | 30.10 | 53.25 | 90.96 | 1.143 |
| Actuation 2 | 22.53 | 44.10 | 85.55 | 1.429 | 28.73 | 49.45 | 82.26 | 1.083 |
| Actuation 3 | 21.90 | 40.88 | 73.57 | 1.264 | 26.03 | 48.04 | 84.60 | 1.219 |
| Average | 22.89 | 43.77 | 82.52 | 1.360 | 28.29 | 50.25 | 85.94 | 1.148 |

TABLE 20
Spray content uniformity by assay of composition #32.

| Composition #32 | T0 (%) | T-15 Days (%) | T-30 Days (%) |
|---|---|---|---|
| Assay 1 | 101.23 | 98.87 | 100.32 |
| Assay 2 | 100.28 | 101.57 | 100.32 |
| Assay 3 | 98.79 | 98.89 | 98.95 |
| Assay 4 | 100.44 | 99.82 | 97.69 |
| Assay 5 | 100.08 | 99.49 | 99.30 |
| Assay 6 | 100.08 | 100.93 | 96.40 |
| Assay 7 | 101.58 | 101.31 | 98.56 |
| Assay 8 | 99.47 | 100.20 | 99.16 |
| Assay 9 | 100.75 | 100.12 | 100.39 |
| Assay 10 | 96.34 | 101.32 | 97.56 |
| Average | 99.90 | 100.25 | 98.86 |

What is claimed is:

1. A liquid rizatriptan composition comprising rizatriptan or a salt thereof and a solvent selected from the group consisting of water, ethanol, propylene glycol, polyethylene glycol 400 and a combination thereof.

2. The composition of claim 1, further comprising one or more agents selected from the group consisting of a solubilizing agent, a stabilizer, a permeation enhancer, a preservative, a pH modifier, a sweetener and a flavoring agent.

3. The composition of claim 2, wherein the one or more agents comprises a solubilizing agent selected from a polysorbate and an inclusion complex forming agent.

4. The composition of claim 2, wherein the one or more agents comprises a stabilizer selected from the group consisting of D,L-alpha tocopherol, butylated hydroxytoluene, methionine, ascorbyl palmitate, ascorbic acid, butylated hydroxyanisole, citric acid, ethylenediamine tetra acetic acid, sodium bisulfate, tert-butylhydroquinone, propyl gallate and a combination thereof.

5. The composition of claim 2, wherein the one or more agents comprises a permeation enhancer selected from the group consisting of octanoic acid, oleic acid, polysorbate 80, menthol, EDTA, sodium edetate, cetylpyridinium chloride, sodium lauryl sulfate, citric acid, sodium desoxycholate, sodium deoxyglycolate, glyceryl oleate, L-lysine and a combination thereof.

6. The composition of claim 2, wherein the one or more agents comprises a preservative selected form the group consisting of benzalkonium chloride, butyl paraben, methyl paraben, ethyl paraben, propyl paraben, sodium benzoate, benzoic acid and a combination thereof

7. The composition of claim 1, wherein the composition has a pH from about 3 to about 11.

8. The composition of claim 2, wherein the one or more agents comprises a pH modifier selected from the group consisting of dilute hydrochloric acid, citric acid, fumaric acid, lactic acid, dilute sodium hydroxide, sodium citrate, sodium bicarbonate, sodium carbonate, ammonium carbonate and a combination thereof.

9. A liquid rizatriptan composition comprising:
from about 2% to about 15.73% w/w rizatriptan; and
a solvent selected from the group consisting of from about 1% to about 97% w/w water, from about 10% to about 60% w/w ethanol, from about 1% to about 50% w/w propylene glycol, from about 1% to about 50% polyethylene glycol 400 and a combination thereof,
wherein w/w denotes weight by total weight of the formulation.

10. The composition of claim 9, further comprising from about 10% to about 50% w/w of a solubilizing agent selected from the group consisting of a polysorbate and a cyclodextrin.

11. The composition of claim 10, wherein the cyclodextrin is selected from the group consisting of hydroxypropyl beta cyclodextrin and sulfobutylether beta cyclodextrin.

12. The composition of claim 9, further comprising from about 0.002% to about 0.2% w/w of a stabilizer.

13. The composition of claim 9, further comprising from about 0.02% to about 7.8% w/w of a permeation enhancer.

14. The composition of claim 9, further comprising from about 0.005% to about 0.2% w/w of a preservative.

15. A liquid rizatriptan composition comprising:
about 7.414% w/w rizatriptan benzoate;
about 20% w/w ethanol; and
about 72.394% w/w water,
wherein w/w denotes weight by total weight of the composition.

16. The composition of claim 15, further comprising about 0.002% w/w butylated hydroxytoluene, about 0.02% w/w butylated hydroxy anisole, about 0.05% w/w edetate disodium and about 0.02% w/w benzalkonium chloride.

17. The composition of claim 15, wherein the composition provides a pharmacokinetic parameter following nasal administration to a beagle dog selected from the group consisting of a Cmax from about 100 to about 650 nanograms per milliliter, a Tmax from about 3 to about 20 minutes, an AUC from 3,500 to about 17,000 nanogram minutes per milliliter and a combination thereof.

18. A method of treating migraines comprising administering to a subject in need thereof an effective amount of a composition of claim 1.

19. The method of claim 1, wherein the composition is administered nasally via a spray pump at an amount from about 50 to about 200 microliters.

20. A liquid rizatriptan composition comprising:
about 14.534% w/w rizatriptan benzoate;
about 50% w/w ethanol; and
about 29.346% w/w water,
wherein w/w denotes weight by total weight of the composition.

21. The composition of claim 20, further comprising about 0.1% w/w Vitamin E, about 0.02% w/w edetate disodium, dihydrate, about 0.5% w/w menthol and about 0.5% w/w sucralose.
